(19) United States
(12) Patent Application Publication
(10) Pub. No.: US 2007/0116746 A1
(43) Pub. Date: May 24, 2007

(54) Packaging items containing a human pheromone component

(76) Inventors: Philip Y. Braginsky, Forest Hills, NY (US); Barry J. Marenberg, New Providence, NJ (US); Katey A. Lang, New York, NY (US)

Correspondence Address: Barry J. Marenberg, 76 Brookside Drive, New Providence, NJ, 07974, US

(21) Appl. No.: 11/376,947

(22) Filed: Mar. 16, 2006

Related U.S. Application Data
(60) Provisional application No. 60/738,890, filed on Nov. 22, 2005.

(51) Int. Cl. A61K 31/56; A61K 9/70

(52) U.S. Cl. 424/443; 514/170

(57) ABSTRACT

Packaging products including a packaging substrate and a human pheromone component are disclosed. The packaging product and incorporated human pheromone components causes a person exposed to the product to experience unique, mood altering effects. The effect may be an overall sense of well-being or one of heightened sexual attractiveness.

### RELATED APPLICATIONS

This application claims the benefit of U.S. Provisional Application No. 60/738,890, filed Nov. 22, 2005.

### FIELD OF THE INVENTION

The present invention relates to packaging items comprising a human pheromone component.

### BACKGROUND

Pheromones

Pheromones are chemicals emitted by living organisms to send messages to individuals of the same species. The classes most widely explored are the sex pheromones produced by female moths which are used to attract conspecific males for mating. Bombykol, the sex pheromone of the silkmoth, was first synthesized in 1959.

Most pheromones consist of blends of two or more chemicals which need to be emitted at exactly the right proportions to be biologically active. The female effluvia or sex gland can contain additional compounds which are related to the pheromone components and whose biological function is often unclear. On the other hand, many attractants of male moths have been discovered simply by field screening. In several cases it could later be shown that the attractant found with this technique was identical to the natural pheromone produced by the female. In most others, the composition of the true pheromone is still unknown.

While it is known that pheromone systems exist in insects, an increasing number of studies have shown that pheromones play a role in many species, including humans. Pheromones in humans are believed to be produced by the apocrine glands. These glands become functional after reaching puberty, which could explain why most people develop an attraction for others at that time. Pheromones could also be the reason why a person can sense “chemistry”, or feel an instant attraction or dislike when first meeting someone.

Certain compounds believed to have pheromone properties affect a specific behavioral or physiological response in human subjects, e.g., a reduction of negative affect, mood, and character traits. In one particular example, nasal administration provides for contacting neurochemical receptors of a heretofore poorly understood neuroendocrine structure, commonly known as the vomeronasal organ (“VNO); also known as “Jacobson's organ”), with one or more steroid(s) or with compositions containing the steroid(s). This organ is accessed through the nostrils of most higher animals—from snakes to humans, and has been associated, inter alia, with pheromone reception in certain species (see generally Muller-Schwarze & Silverstein, Chemical Signals, Plenum Press, New York (1980)). The axons of the neuroepithelia of the vomeronasal organ, located supra palatinal, form the vomeronasal nerve and have direct synaptic connection to the accessory olfactory bulb and indirect input from there to the cortico-medial amygdaloid basal forebrain and hypothalamic nuclei of the brain. The distal axons of terminalis nerve neurons may also serve as neurochemical receptors in the VNO. Stensaas, L. J., et al., J. Steroid Biochem. and Molec. Biol. (1991) 39:553. This nerve has direct synaptic connection with the hypothalamus.

Packaging

Commercial items, materials and products are often packaged in containers or wrappers for handling, storage, and bundling. Many products are typically packaged in some protective container or material. Typical containers for packaging products include various types of rigid containers such as paperboard boxes or open-topped holders in which food is inserted, or flexible wrappers such as sheets of wax paper or other similar materials which can be wrapped around the product. Containers and wrappers may be made out of various materials including but not limited to paper, metal, and plastic.

To manufacture paper, the materials to be used must first be converted into pulp, a concentrated mixture of fibers suspended in liquid. The source of fiber is often natural (softwood trees such as spruce or other plants) or recycled, such as old corrugated boxes, newsprint, or mixed paper.

When natural materials are used to make paper it is mostly the cellulose that is used. This makes it necessary to break down the lignin inside of the plant's cell walls and separate it from the cellulose. This is called pulping and is typically done by well known chemical processes. Mechanical processes can be used but they leave the lignin that causes paper to yellow over time. Therefore mechanical pulps are commonly used for packaging, newsprint, and other low-strength applications. Mechanical pulp may be blended with chemical pulp to produce a middle-level product with low cost and reasonable strength and color properties.

Pulp that is broken down chemically is known as “chemical pulp.” The main purpose of a chemical pulping process is to break down the chemical structure of lignin and render it soluble in a liquid (most often water) so it may be washed from the remaining fibers. Removing the lignin from wood chips also serves to break them apart into the fibers that compose pulp.

Recycled fibers do not need to be pulped in the conventional sense. These fibers have already been treated once, so instead they need a more gentle process to break the fibers apart while preserving their integrity.

Once the fibers have been extracted, they may also be bleached, dyed, or have additional ingredients added to alter the appearance of the final product. For example, Kaolin (or calcium carbonate) is added to produce the glossy papers typically used for magazines.

The pulp mixture is then further diluted up to 99 percent or more with water and/or other fluids resulting in a thin slurry. This slurry is drained through a fine-mesh moving screen to form a fibrous web. This moving web is pressed and dried into a continuous sheet of paper.

In the mould process, a quantity of pulp is placed into a form, with a wire-mesh base, so that the fibers form a sheet on the mesh and excess water can drain away. Pressure may be applied to help remove additional water. The paper may then be removed from the mould, wet or dry, and go on to further processing.

Most mass-produced paper is made using the continuous Fourdrinier process to form a reel or web of fibers in a thin sheet. In commercial paper making processes, the paper fibers are deposited from a pulp slurry onto a Fourdrinier forming wire which supports the deposited fibers to allow formation of the continuous paper web. The forming wire is an endless loop of wire screen which is supported by rollers that are driven to move the forming wire. In the processing of the formed web, the web may be transferred to other support wires or felts and the web may be engaged by press felts, all of which are similarly endless loops that are supported and driven by rollers. Similar forming wires or felts are used in air-laid papermaking.

When dried, this continuous web may be cut into rectangular sheets by slicing the web vertically and horizontally to the desired size. After the paper web is produced, the water must be removed from it in order to create a usable product. This is accomplished through pressing and drying. The methods of doing so vary between the different processes used to make paper, but the concepts remain the same.

Pressing the sheet removes the water by force. Once the water is forced from the sheet, another absorbent material must be used to collect this water. On a paper machine this is called a felt (not to be confused with the traditional felt). When making paper by hand, a blotter sheet is used.

Drying involves using air, heat, and pressure to remove water from the paper sheet. On the paper machine, the most common is the steam-heated can dryer. These dryer cans heat to temperatures above 200° F. and are used in long sequences of more than 40 cans. The heat produced by these can easily dry the paper to less than 6% moisture. (See, http://en.wikipedia.org/wiki/Paper)

After leaving the press section, the sheet encounters these drying cylinders that are large hollow metal cylinders, heated internally with steam that dry the paper as it passes over them. The sheet will be wound up and down over many cylinders in the drying process. Between dryer sections, the paper may be coated with pigments, latex mixtures, or many other substances to give it a higher gloss or to impart some other desirable characteristic. After another round of drying, the paper sheet is passed through a series of polished, close-stacked metal rollers known as a “calender” where it is pressed smooth. Finally, the sheet is collected on a take-up roll and removed from the paper machine. From the headbox, it may have traveled half a mile or more in less than a minute.

Films, coatings, pigments, layers and more may be added to a paper substrate during various stages of its manufacture. These can be used to impart specific desired properties to the paper. Paper has been used as a delivery mechanism for powders, fragrances and other substances.

Packaging cartons or boxes can be made from many materials, and in fact are often a composite. Cardboard is a common packaging material that is a heavy wood-based type of paper, notable for its stiffness and durability. Most common is the corrugated cardboard, made by folding cardboard into a rough semblance of a sine wave, and then pressing it between two more pieces of cardboard. Because of the triangular shape of the cavities, the addition of corrugation greatly increases the strength of the container. Often, cartons are made out of a single piece of cardboard, when the strength of corrugated cardboard isn't needed. This cardboard may be waxed to form a moisture barrier. This may serve to contain a liquid product or keep a powder dry. Other layers, films or other substances maybe added either during the manufacture of the card stock or during the manufacture of the carton.

Polymer materials including nylon are another common packaging material since they can be molded in to a container of almost any shape. These plastics have a number of attractive properties that are desirable for use in packaging including toughness, high-strength, clarity, and flexibility. One of the prior art techniques for the formation of packaging films having a nylon layer is the high-temperature or “hot” extrusion coating process in which a nylon resin is heated to a high temperature and extruded while still in a molten condition directly onto a substrate. A process of this type is shown in U.S. Pat. No. 3,570,748.

Metal is also used as a packaging material for products and substances. Metal foil is made by passing coiled aluminum or other metal sheets repeatedly between steel rollers, set progressively closer together. Each pass squeezes the foil a little thinner. Aluminum foil finds its largest use in containers and packaging for food, cosmetics and pharmaceutical products. Aluminum withstands both heat and cold. It is easy to sterilize for food and medical applications and is an excellent barrier against liquids, vapors and light. It is also non-toxic and imparts no taste or odor, is an excellent electrical conductor and transmits conducted heat, and reflects radiant heat.

Aluminum Oxide, which forms on the surface of all aluminum metal in the presence of air, is stable in the pH range of 4.5-8.5, making aluminum suitable for storage of many different food types. Aluminum beverage cans and food cans have a protective polymer coating applied on the inside to prolong storage life and for other purposes. Other substances made be mixed with a metal or provided as a composite in the form of a layer, film or other form with the metal to impart various purposes including fragrances and flavors.

Packaging of any material including paper, polymer or metal based materials is often produced from a packaging laminate or composite with a plurality of layers which each afford specific properties to the packaging. For example, such packaging normally include outer layers possessing superior thermosealing properties in order to make for reforming into packaging containers by fold formation and sealing of the outer layers against one another. Such thermosealable layers normally consist of thermoplastic which also functions as liquid and moisture barrier, on the one hand preventing moisture from the ambient surroundings from penetrating into the packaging wall and into the packed food product, and, on the other hand, preventing the liquid food from leaking out through the packaging wall into the surroundings. Such packaging laminates often also include a rigidifying core layer in order to impart to the finished packaging container strength and configurational stability, normally of paper or paperboard. Core layers of thicker, homogeneous or foamed plastic layers are also common.

Adhesives are used with many packaging. Often times various compounds may be suspended in adhesives prior to their application in a packaging product.

In packaging, a wrapper is typically a flat sheet often made out of paper, cellophane, metal foil or plastic which is folded or sealed to wholly or partially enclose an object and protect it from spoilage, handling, moisture, air and other elements.

### SUMMARY OF THE INVENTION

The invention provides packaging items that are formulated with and/or coated with a human pheromone substance. The pheromone will be carried by the packaging as a delivery mechanism to a human subject and specifically the VNO of a human subject. The packaging items of the present invention will cause a person coming into contact with the packaging item to experience a unique, mood-altering effect. This effect may also be an overall sense of well-being or may be one of heightened sexual attractiveness.

The invention relates to all types of packaging items including but not limited to paper, plastic and metal-based (metallic) substrates as well as adhesives, fasteners and other items associated with packaging of items or substances. The packaging may be intended for food items, personal care products, pharmaceuticals, drugs and other therapeutics, home decorations, magazines, books, newspapers, electronics, and paper products.

### DESCRIPTION OF THE INVENTION

As used herein, a “pheromone” is a substance that provides chemical means of communication between members of the same species through secretion and peripheral chemoreception. In mammals pheromones are usually detected by receptors in the vomeronasal organ of the nose. Commonly, pheromones effect development, reproduction and related behaviors. The pheromone compositions according to the present invention are intended to cause a change in human hypothalamic function, thereby altering certain behavior and physiology mediated by the hypothalamus of individuals.

In addition to physiological responses, pheromones can be identified by their species specific binding to receptors in the vomeronasal organ (VNO). Thus, human pheromones bind to human receptors. This can be demonstrated by measuring the change in the summated potential of neuroepithelial tissue in the presence of the pheromone. Human pheromones induce a change of at least about −5 millivolts in human neuroepithelial tissue of the appropriate sex (the binding of pheromones is generally sexually dimorphic, i.e., different in the effect of, or response to, a compound or composition between males and females of the same species). Human pheromones induce sexually dimorphic changes in receptor binding potential in vivo in the human VNO. Naturally occurring human pheromones can be extracted and purified from human skin and they can also be synthesized. “Human pheromones” are pheromones which are naturally occurring in humans and effective as a specifically binding ligand in human VNO tissue, regardless of how the pheromone was obtained. Thus, both a synthesized and purified molecule may be considered a human pheromone.

The present invention provides the application of human pheromone compositions to a substrate or the incorporation of human pheromone compositions into a substrate. In one embodiment one or more pheromone compositions are deposited on a substrate. Additionally, a film forming agent may serve to microencapsulate the composition and bond it to the substrate. When the film forming agent dries, it protects the pheromone composition. A cover sheet may then cover the film forming agent.

The pheromone composition may be deposited by any known method including but not limited to dipping, brushing, spraying, rolling, depositing, laminating and extruding. The pheromone composition may also be deposited between layers of a composite package and may be suspended in or provided in a matrix of an adhesive, ink, color solution or other component applied to or incorporated within packaging. Therefore, in another embodiment one or more pheromone compositions are incorporated into a packaging composition.

In the packaging industry conventional substrates include but are not limited to paper wrappers, a cardboard or a paper box, foil or metal layers, polymers films or boxes. Different applications and performance standards require different substrates. In addition it is contemplated that any or all of these materials may be combined using know methods, including but not limited to layers, to create a composite package.

For paper and cardboard packaging the pheromone composition may be incorporated into the substrate during its manufacture or absorbed by it after manufacture. During manufacture the pheromone composition may be included at any point during the manufacture. Preferably this is done once the fibers have been extracted and are in a slurry or while drying the paper. The paper may be coated with pigments, latex mixtures, and many other substances including the pheromone composition to give it different finishes and to impart other desirable characteristic. After manufacture spraying, dipping, brushing, depositing, rolling, laminating or other known methods of applications may be used to apply the pheromone to the substrate for its complete or partial absorption. Preferably the pheromone is sprayed onto the substrate.

Polymer packaging takes many forms including but not limited to polymer films, plastic boxes, and polystyrene films or foams, and liners. Polymer packaging films come as single wound sheeting, centerfold, tubing, monolayer, co-extrusions, and laminations (adhesive and extrusion). Polymer materials typically include but are not limited to Low-Density Polyethylene (LDPE), Linear Low Density Polyethylene (LLDPE), High Density Polyethylene (HDPE), Coaxially Oriented Polypropylene (COPP), Biaxially Oriented Polypropylene (BOPP), Polyethylene Terephthalate (PET), polymide resins such as nylon, Ethylene Vinyl Alcohol Polymer (EVOH), and metallized versions of the same.

Metal foil may be aluminum foil or other metal or metal alloys. As with paper based packaging materials, the pheromone composition may be incorporated into a plastic or metal packaging substrate at the time of manufacture or applied to the substrate after manufacture.

Often packaging comprises one or more layers in a composite. For paper and cardboard packaging, the packaging may be covered by a polymeric plastic film or has a separate polymeric film sealed around the contents and inserted into the cardboard packaging avoiding undesired conditions such as odors, gases or liquids passing from the packaged item to the outside and vice-versa, when the packaged item is food for instance. This plastic film (for example, polyvinylidene chloride) acts as a barrier against these substances.

In the case of food packaging in particular, it is important that the packaged food retain the integrity of its taste and odor during storage. To do this, the sealant layer should have good organoleptic properties and should not capture the food specific taste and odor. It is also often important that the taste and odor of the food are not altered, either by addition or by scalping, by the sealant layer. In addition, it is important that the packaging film has good barrier properties against oxygen and moisture or water vapor to preserve the goods over a long period of time.

One embodiment of the invention is a multilayer packaging comprising at least one cellulosic fibrous material, or metal foil material or a polymer based material substrate layer, and at least one polymeric film layer coated on the substrate, wherein at least one of these layers comprises a human pheromone component. The polymeric film may comprise at least one barrier layer comprising an olefin polymer, and at least one sealant layer comprising an amorphous polyamide, the sealant layer being in contact with the packaged product.

The packaging of this embodiment of the invention has excellent barrier properties to oxygen and water vapor. The packaging has a good sealability on itself, and it also has good organoleptic properties. Food packaged in the package of the present invention does not lose its taste or its odor. Further, the additive of the invention has no influence on the taste and odor of the packaged food.

Moreover, the packaging of the invention as a film can be transparent. As such it is invisible to the customer, thus providing a packaging material that has the appearance of the paper or board substrate or the plastic substrate when used as a composite of providing a view of the item that is packaged.

There are other multilayer embodiments known in the art and provided by the invention. The multilayer packaging may comprise at least one cellulosic fibrous material, or metal foil material or a plastic material substrate layer, and at least one layer on the substrate, wherein at least one of these layers comprises a human pheromone component.

The packaging of the invention can find use in food packagings. It can find use in the packaging of solid food like chocolates, biscuits, and also in liquid food like fruit juices, beverages, soups, oils, water, and alcohols. Further, the invention can be used to package non-food items including but not limited to paper products, clothing, fashion accessories, furniture, sporting goods, electronics, personal care products, drugs, and others.

In a further embodiment of the invention the pheromone component may be incorporated into a transparent polypropylene film, produced by coextrusion and coated on one or both sides, for twist wrapping. Twist wrapping is a particular method of closing complete wrappings for packaging of goods. This packaging method is particularly known in connection with the complete wrapping of relatively small goods items, including round or approximately round articles such as candies, bottles, candles, rolls of circular candies, chocolate bars, marzipan bars or the like. Important for the use of twist wrapping is the suitability of the film, which preferably exhibits neither tear-starting nor tearing-off at the twist points, but on the other hand must be sufficiently stiff so that no shrinkage or crumpling occurs during twisting.

According to the state of the art, it is predominantly cellophane, i.e. regenerated cellulose, non-oriented polypropolene or PVC film which are employed for candy wrappings. See, e.g., “Verpacken mit Kunststoffen” [“packing with plastics”] by Guenther Kuehne, published 1974, Carl-Hanser-Verlag, Munich, page 63. The invention may also provide a film of the initially mentioned generic type, of which the characterizing features are the polypropylene substrate layer additionally containing a low molecular weight hydrocarbon resin. The pheromone component may be incorporated into the cellophane, may be an additional layer, may be incorporated into the substrate layer or other known embodiments.

The present invention provides for packaging comprising a pheromone component. The packaging may be paper based, polymer based (i.e. polystyrene, thin polymer film), or metal based (i.e. foil). The pheromone component may be an added layer or incorporated into the base or other layers. Although specific embodiments have been illustrated and described herein for purposes of description of the preferred embodiment, it will be appreciated by those of ordinary skill in the art that a wide variety of alternate and/or equivalent implementations calculated to achieve the same purposes may be substituted for the specific embodiments shown and described without departing from the scope of the present invention. Therefore, it is manifestly intended that this invention be limited only by the claims and the equivalents thereof. In the detailed description of the preferred embodiments, reference is made to the accompanying drawings, which form a part hereof, and in which is shown by way of illustration, specific embodiments in which the invention may be practiced. It is to be understood that other embodiments may be used, and structural or logical changes may be made, without departing from the scope of the present invention. The detailed description is therefore not to be taken in a limiting sense, and the scope of the present invention is defined by the appended claims.

The present invention is not limited in the pheromone composition used. Exemplary pheromone compounds include, but are not limited to, copulins (such as short chain aliphatic acids), estrene steroids, vomeropherins, such as 19-nor-cholane steroids, 19-nor-pregnane steroids, androstadienone, androstane, androstene and androstenedione compounds, as well as combinations thereof and metabolites thereof.

Metabolites of the above-recited human pheromone compounds are also contemplated.

Upon the VNO sensing of the pheromone in the packaging in accordance with the present invention, the human pheromone will exert its effect by altering hypothalamic function of an individual. Such alteration of hypothalamic function will bring about physiological and behavioral functions of individuals which are normally regulated by the hypothalamus. The changes in physiological functions may cause the individual exposed to the packaging product to experience a unique, mood-altering effect or an overall sense of well-being and/or heightened sexual attractiveness.

It has been found that certain human pheromones have a distinct effect on men and others on women, i.e., the compounds are sexually dimorphic. As such, the present invention is not limited to the use of a specific pheromone compound within the packaging product and it is contemplated that certain packaging products directed for men will contain a human pheromone compound different from the packaging product directed for women or that a single product may contain a mix of human pheromone compounds.

1. A packaging product comprising:
a packaging substrate; and
a pheromone component.

2. A packaging product as in claim 1 wherein:
the substrate is substantially paper.

3. A packaging product as in claim 2 wherein:
the paper is cardboard.

4. A packaging product as in claim 1 wherein:
the substrate is substantially a polymer.

5. A packaging product as in claim 4 wherein:
the polymer is polystyrene.

6. A packaging product as in claim 4 wherein:
the substrate is nylon.

7. A packaging product as in claim 4 wherein:
the substrate is a material selected from the group consisting of Low-Density Polyethylene (LDPE), Linear Low Density Polyethylene (LLDPE), High Density Polyethylene (HDPE), Coaxially Oriented Polypropylene (COPP), Biaxially Oriented Polypropylene (BOPP), Polyethylene Terephthalate (PET), polymide resins such as nylon, Ethylene Vinyl Alcohol Polymer (EVOH), and metallized versions of the same.

8. A packaging product as in claim 1 wherein:
the substrate is substantially metal.

9. A packaging product as in claim 4 wherein:
the metal is a metal foil.

10. A packaging product as in claim 1 wherein:
the substrate is a combination of materials selected from the group consisting of paper, polymer and metal.

11. A packaging product as in claim 1 wherein:
the pheromone component comprises a human pheromone.

12. A packaging product as in claim 11 wherein:
the pheromone component comprises two or more human pheromone compounds.

13. A packaging product as in claim 11 wherein:
the human pheromone compound is selected from the group consisting of 19-nor-cholane compounds, 19-nor-pregnane compounds, estrene steroids, short chain aliphatic acids, androstadienone steroids, androstene, androstane and androstenedione compounds, combinations thereof and metabolites thereof.

14. A packaging product as in claim 1 wherein:
the pheromone component is coated on the substrate.

15. A packaging product as in claim 14 wherein:
the coating is applied by a method selected from the group consisting of dipping, brushing, spraying, rolling, depositing, laminating and extruding.

16. A packaging product as in claim 1 wherein:
the pheromone component is incorporated into the substrate during manufacture of the substrate.

17. A packaging product as in claim 16 wherein:
the substrate is substantially a material selected from the group consisting of paper, polymer, and metal.

18. A packaging product as in claim 1 further comprising:
an adhesive applied to the substrate.

19. A packaging product as in claim 18 wherein:
the pheromone component is incorporated into the adhesive.

20. A method of manufacturing a packaging product comprising:
forming a substrate;
providing a layer on the substrate comprising a pheromone component;
forming the substrate with the layer into a package to carry an item.

21. A method of manufacturing a packaging product as in claim 20 wherein:
the substrate is substantially paper.

22. A method of manufacturing a packaging product as in claim 20 wherein:
the substrate is substantially polymer based.

23. A method of manufacturing a packaging product as in claim 20 wherein:
the substrate is substantially metallic.

24. A method of manufacturing a packaging product as in claim 20 wherein:
the pheromone component is a human pheromone.

25. A method of manufacturing a packaging product as in claim 24 wherein the human pheromone is selected from the group consisting of: 19-nor-cholane compounds, 19-nor-pregnane compounds, estrene steroids, short chain aliphatic acids, androstadienone steroids, androstene, androstane and androstenedione compounds, combinations thereof and metabolites thereof.

26. A method of manufacturing a packaging product comprising:
forming a substrate comprising a pheromone component;
forming the substrate into a package to carry an item.

27. A method of manufacturing a packaging product as in claim 25 wherein:
the pheromone component is a human pheromone.

28. A method of manufacturing a packaging product as in claim 27 wherein the pheromone component is selected from the group consisting of: 19-nor-cholane compounds, 19-nor-pregnane compounds, estrene steroids, short chain aliphatic acids, androstadienone steroids, androstene, androstane and androstenedione compounds, combinations thererof and metabolites thereof.
